(19) United States
(12) Patent Application Publication
(10) Pub. No.: US 2025/0161545 A1
(43) Pub. Date: May 22, 2025

(54) Hydraulic Block for Dialysis, Hydraulic System for Dialysis and Method for Manufacturing Hydraulic Block

(71) Applicant: Fresenius Medical Care Deutschland GmbH, Bad Homburg (DE)

(72) Inventors: Xibin Li, Bad Homburg (DE); Hao Zhang, Bad Homburg (DE); Maosong Wu, Bad Homburg (DE)

(21) Appl. No.: 18/841,746

(22) PCT Filed: Feb. 28, 2022
(86) PCT No.: PCT/CN2022/078354

§ 371 Date: Aug. 27, 2024

(51) Int. Cl. A61M 1/16; A61M 1/36

(52) U.S. Cl. CPC A61M 1/1629; A61M 1/3627; A61M 2206/16

(57) ABSTRACT

A hydraulic block for dialysis comprises: a base body formed with a fluid accommodating cavity; and at least one standing structure standing on the base body, wherein the standing structure comprises at least one vertical fluid cavity fluidly connected with the fluid accommodating cavity. Also disclosed are a corresponding hydraulic system for dialysis and a corresponding method for manufacturing the hydraulic block. According to exemplary embodiments of the present disclosure, both flow paths and chambers are integrated into a molded plastic hydraulic block to further improve integration and reduce the number of parts to be assembled. The hydraulic block can be molded with some installation interfaces so as to allow for easy and quick mounting of some functional components. Further, the hydraulic block can be disinfected and then used repeatedly.

### CROSS-REFERENCE TO RELATED APPLICATIONS

The present application is the national stage entry of International Patent Application No. PCT/CN2022/078354, filed on Feb. 28, 2022, the disclosure of which is incorporated herein by reference.

### TECHNICAL FIELD

The present disclosure relates to a hydraulic block for dialysis, e.g., hemodialysis, a corresponding hydraulic system for dialysis and a corresponding method for manufacturing the hydraulic block.

### BACKGROUND ART

A dialysis treatment is a procedure for removing toxic substances and metabolites normally removed by the kidneys, and for aiding in regulation of fluid and electrolyte balance.

The dialysis treatment may be carried out by various types of dialysis procedures, such as a hemodialysis (HD) and a peritoneal dialysis (PD). The hemodialysis is usually executed by using a hemodialysis machine.

The known hemodialysis machine usually has a considerable weight and size mainly because of its complex and bulky hydraulic system. The hydraulic system includes a large number of flow paths and other functional components to achieve dialysate proportioning, delivering and/or balancing.

As a typical hydraulic system, separate components, such as chambers, pumps, valves, sensors and so on, are mounted on a metal bracket, and many tubes are used for connecting the components together to achieve a corresponding fluid flow system. The manufacturing or assembling process for this kind of hydraulic system is quite labor intensive and thus it is difficult to facilitate an automatic assembly process. Moreover, there is a big leakage and pollution risk because there are too many detachable tubes and connections. Therefore, maintenance and diagnostic efforts are quite big for such a hydraulic system in use of the HD machine.

To this end, some integrated hydraulic cassettes are proposed, in which the flow paths are integrated into the hydraulic cassettes so as to reduce the complexity of routing of the tubes. However, such hydraulic cassettes are usually disposable and thus can only be used once so that they must be replaced after each dialysis treatment, which will significantly increase the cost of dialysis treatment.

Further, the known integrated hydraulic cassettes still have problems of low integration and troublesome manufacturing process.

Thus, there still is a need to make further improvements.

### SUMMARY OF THE DISCLOSURE

An aspect of the present disclosure is to provide an improved hydraulic block for dialysis, a corresponding hydraulic system for dialysis and a corresponding method for manufacturing the hydraulic block.

According to a first aspect of the present disclosure, provided is a hydraulic block for dialysis, comprising: a base body formed with a fluid accommodating cavity; and at least one standing structure standing on the base body, wherein and the standing structure comprises at least one vertical fluid cavity fluidly connected with the fluid accommodating cavity.

According to an optional embodiment of the present disclosure, the standing structure and the base body are molded integrally; or the standing structure and the base body are molded individually so that the standing structure can be fitted onto the base body. or at least one standing structure is molded individually, and the base body and the rest of the standing structure are molded integrally, so that the at least one standing structure can be fitted onto the base body.

According to an optional embodiment of the present disclosure, the fluid accommodating cavity initially opens at a side of the base body after molding of the base body; and/or the fluid accommodating cavity comprises a flow path; and/or the hydraulic block is configured for hemodialysis; and/or the hydraulic block is configured to prepare and/or deliver dialysate; and/or the hydraulic block is configured to be reusable, e.g., after being disinfected; and/or the standing structure is configured as at least one of a water inlet chamber, a heating chamber for heating water, a degassing chamber for water, and an air separation chamber for a used dialysate; and/or the standing structure and the base body are molded from at least one of polyethersulfone (PES), polyphenylene oxide (PPO), and polyphenylsulfone (PPSU).

According to an optional embodiment of the present disclosure, the base body is configured as a flat body; and/or the standing structure and the base body are molded by an injection molding process.

According to an optional embodiment of the present disclosure, the standing structure and the base body are molded as a single plastic piece; and/or the standing structure and the base body are molded by a one-spot injection molding process.

According to an optional embodiment of the present disclosure, the at least one vertical fluid cavity comprises at least one vertical fluid chamber and at least one vertical flow channel which are in fluid communication with each other via a fluid communication path; and/or the base body is molded with at least one installation interface for at least one functional component for dialysis; and/or the base body is molded with a mixing structure for preparing dialysate; and/or the hydraulic block comprises a cover fixedly connected to the base body to close the fluid accommodating cavity.

According to an optional embodiment of the present disclosure, the vertical flow channel is located outside of the vertical fluid chamber and adjacent to a vertical wall of the vertical fluid chamber; and/or the vertical flow channel is partially defined by a corresponding portion of the vertical wall of the vertical fluid chamber; and/or the vertical fluid chamber is molded by a first columnar core, for example a cylindrical core, on a mold; and/or the vertical flow channel is molded by at least one second columnar core, for example a cylindrical core, on the mold; and/or the standing structure is molded with an outward opening directly leading to the fluid communication path; and/or the mixing structure comprises at least one mixing chamber, a fluid outlet opening into the mixing chamber, and at least two fluid inlets each opening into the mixing chamber; and/or the cover is a plastic cover.

According to an optional embodiment of the present disclosure, the vertical flow channel is molded onto the vertical fluid chamber; and/or the vertical fluid chamber is molded by positioning the first columnar core on one side of the mold; and/or the vertical flow channel is molded by positioning one second columnar core on each of two sides of the mold; and/or the at least one mixing chamber comprises a first chamber and a second chamber fluidly connected to the first chamber by a flow communication passage and located downstream of the first chamber, and the at least two fluid inlets comprise a first fluid inlet for a first fluid, a second fluid inlet for a second fluid, and a third fluid inlet for a third fluid, wherein the first fluid inlet opens tangentially into the first chamber, the second fluid inlet opens into the first chamber, the third fluid inlet opens into the second chamber, and the fluid outlet opens into the second chamber; and/or the cover is bonded to the base body by a plastic bonding process, for example at least one of an ultrasonic welding process, a diffusion bonding process, an infrared welding process, a resistive welding process and a laser welding process; and/or the outward opening is configured to be closeable and/or to be fluidly connected with an external part.

According to an optional embodiment of the present disclosure, the second fluid inlet is located downstream of the first fluid inlet; and/or the second fluid inlet is oriented toward a center of the first chamber; and/or the third fluid inlet and/or the fluid outlet is oriented toward a center of the second chamber; and/or the first chamber and/or the second chamber is configured as a circular chamber; and/or the mixing structure is configured so that a first swirling fluid flow can be generated within the first chamber; and/or the mixing structure is configured so that a second swirling fluid flow can be generated within the second chamber; and/or the flow communication passage is configured in an arc shape.

According to an optional embodiment of the present disclosure, the first swirling fluid flow and the second swirling fluid flow have the same swirling direction; and/or the flow communication passage is configured to be bent outwards.

According to an optional embodiment of the present disclosure, the mixing structure is configured so that a first fluid flow direction into the first chamber via the first fluid inlet is opposite to a second fluid flow direction into the first chamber via the second fluid inlet; and/or the flow communication passage opens tangentially into the first chamber; and/or the flow communication passage opens tangentially into the second chamber; and/or the mixing structure is configured so that a third fluid flow direction into the flow communication passage from the first chamber is opposite to the first fluid flow direction into the first chamber via the first fluid inlet and/or parallel to the second fluid flow direction into the first chamber via the second fluid inlet; and/or the mixing structure is configured so that a fourth fluid flow direction into the second chamber via the flow communication passage is opposite to the third fluid flow direction into the flow communication passage from the first chamber and/or parallel to a fifth fluid flow direction into the second chamber via the third fluid inlet; and/or the mixing structure is configured so that a sixth fluid flow direction out of the second chamber via the fluid outlet is substantially perpendicular to the fifth fluid flow direction into the second chamber via the third fluid inlet.

According to an optional embodiment of the present disclosure, the second fluid inlet and/or the third fluid inlet is molded with a narrowed orifice; and/or the first fluid is water, e.g., reverse osmosis water, and at least one of the second fluid and the third fluid is concentrate required for preparing dialysate, for example bicarbonate.

According to an optional embodiment of the present disclosure, the narrowed orifice is molded by a first slider located at a first side of the narrowed orifice facing toward the first chamber or the second chamber and a second slider located at a second side of the narrowed orifice opposite to the first side.

According to an optional embodiment of the present disclosure, the first chamber and/or the second chamber is molded by the first slider and a chamber molding core cooperating with the first slider.

According to an optional embodiment of the present disclosure, during a demolding process, the chamber molding core is drawn in a drawing direction and then the first slider is pulled away in a pulling direction different from, e.g., perpendicular to, the drawing direction.

According to an optional embodiment of the present disclosure, the fluid communication path is located at a top of the standing structure so as to allow fluid to flow into or out of a top of the vertical fluid chamber via the vertical flow channel; and/or the outward opening is oriented upwards; and/or the outward opening can be closed by a sealing structure, e.g., a cap which can be mounted or bonded at the outward opening; and/or the first columnar core has a draft angle of 1-3 degrees; and/or the second columnar core has a draft angle of about 0.5 degrees; and/or the second columnar cores are connected at a middle position of the vertical flow channel to be molded.

According to an optional embodiment of the present disclosure, the cap is configured to be mounted in a form-fitting manner, for example in a snap-fitting manner, and/or by using a fastener, for example a screw, or to be bonded by welding.

According to an optional embodiment of the present disclosure, a welding structure is provided to facilitate welding, e.g., laser welding, between the cover and the base body; and/or the cover is formed at least partially from a material transparent to laser; and/or the base body is formed at least partially from a non-transparent material; and/or the installation interface is configured as a protruding seat, e.g., lower than the standing structure; and/or the at least one functional component comprises at least one of tubes, pumps, valves and sensors.

According to an optional embodiment of the present disclosure, the welding structure comprises a first welding portion, for example one of a groove and a rib, formed at a site to be welded of the cover, and a second welding portion, for example the other of the groove and the rib, formed at a site to be welded of the base body and configured to cooperate with the first welding portion to form a welding seam; and/or the installation interface is configured as a quick connector.

According to an optional embodiment of the present disclosure, the first welding portion is configured as the groove and the second welding portion is configured as the rib; and/or a height of the rib is greater than a depth of the groove, for example by a 0.5-1 mm, before welding; and/or the rib and the groove are located adjacent to an edge to be sealed of the fluid accommodating cavity.

According to a second aspect of the present disclosure, further provided is a hydraulic system for dialysis, wherein the hydraulic system comprises the hydraulic block described above and at least one functional component mounted on the hydraulic block.

According to a third aspect of the present disclosure, further provided is a method for manufacturing the hydraulic block described above, wherein the method comprises molding the hydraulic block by using a mold.

According to exemplary embodiments of the present disclosure, both the flow paths and chambers are integrated into a molded plastic hydraulic block to further improve integration and reduce the number of parts to be assembled. The hydraulic block can be molded with some installation interfaces so as to allow for easy and quick mounting of some functional components. Further, the hydraulic block can be disinfected and then used repeatedly.

### BRIEF DESCRIPTION OF THE DRAWINGS

The present disclosure and advantages thereof will be further understood by reading the following detailed description of some exemplary embodiments with reference to the drawings in which:

FIG. 1 shows a perspective view of a hydraulic block according to an exemplary embodiment of the present disclosure.

FIG. 2 shows a perspective view of the hydraulic block as shown in FIG. 1, as viewed from a different viewing point, to present the other side of the hydraulic block.

FIG. 3 shows a perspective view of a standing structure of the hydraulic block according to an exemplary embodiment of the present disclosure, which is fitted with a corresponding sealing structure.

FIG. 4 shows a sectional view of the standing structure as shown in FIG. 3, with the sealing structure.

FIG. 5 shows a perspective view of the hydraulic block when some functional components have been mounted on an installation interfaces of the hydraulic block.

FIG. 6 shows a perspective view of the hydraulic block when a cover is to be fixedly connected to a base body of the hydraulic block.

FIG. 7 shows a sectional view for illustrating a welding structure for facilitating welding of the cover and the base body according to an exemplary embodiment of the present disclosure.

FIG. 8 shows a sectional view for illustrating the welding structure as shown in FIG. 7 after welding, wherein a welding seam is formed so as to securely fix the cover to the base body.

FIG. 9 shows a sectional view for illustrating a mixing structure for preparing dialysate according to an exemplary embodiment of the present disclosure.

FIG. 10 shows a partially enlarged sectional view of the mixing structure as shown in FIG. 9.

FIG. 11 schematically shows how to mold a first chamber and/or a second chamber of the mixing structure according to an exemplary embodiment of the present disclosure.

### DETAILED DESCRIPTION

Some exemplary embodiments of the present disclosure will be described hereinafter in more details with reference to the drawings to better understand the basic concept of the present disclosure.

The present disclosure mainly relates to a novel hydraulic block, e.g., for hemodialysis,

According to a first aspect of the present disclosure, proposed is a hydraulic block for dialysis, e.g., hemodialysis, comprising: a base body formed with a fluid accommodating cavity; and at least one standing structure standing on the base body, wherein the standing structure and the base body are molded integrally. for example by using an injection molding process, and the standing structure comprises at least one vertical fluid cavity fluidly connected with the fluid accommodating cavity. As an another embodiment, the standing structure and the base body are molded individually so that the standing structure can be fitted onto the base body, which may be advantageous to simplify and/or optimize the manufacture process and cost efficiency if the standing structure and/or the base body has such a configuration that it is difficult or unable to mold them integrally under certain circumstance. The person skilled in art shall understand an alternative embodiment could be also conceived of according to the disclosure that at least one standing structure can be molded individually, while the rest of the standing structure can be molded integrally together with the base body; and then the at least one standing structure is eventually assembled on the base body.

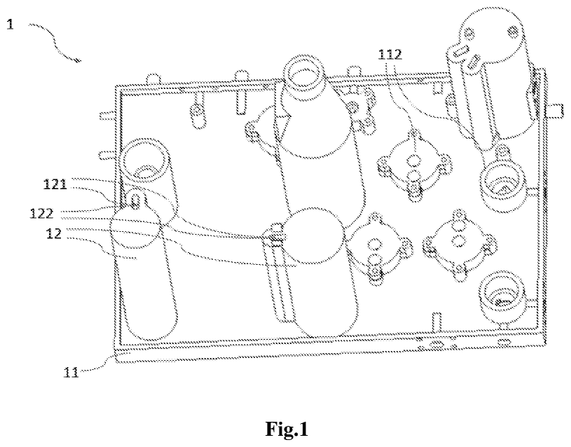

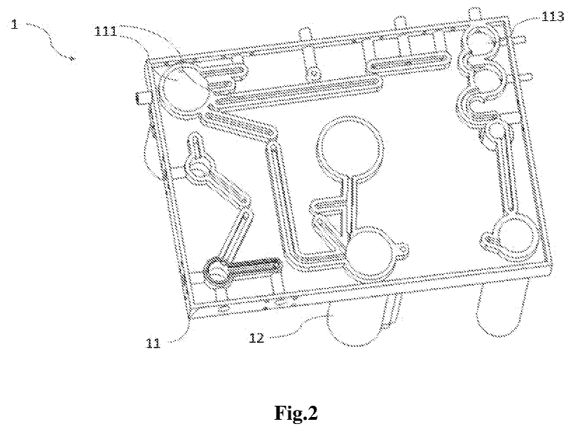

FIG. 1 shows a perspective view of the hydraulic block 1 according to an exemplary embodiment of the present disclosure. FIG. 2 shows a perspective view of the hydraulic block 1 as shown in FIG. 1, as viewed from a different viewing point, to present the other side of the hydraulic block 1.

As shown in FIG. 1 and FIG. 2, the hydraulic block 1 comprises the base body 11 formed with the fluid accommodating cavity 111, in which fluid can flow and be received, and the at least one standing structure 12 standing on the base body 11. The standing structure 12 may stand upright on the base body 11. The standing structure 12 and the base body 11 are molded integrally as a single piece, e.g., a single plastic piece, so that a corresponding fitting process can be omitted. Further, the standing structure 12 is molded with at least one vertical fluid cavity 121 fluidly connected with the fluid accommodating cavity 111, which means that at least a portion of the vertical fluid cavity extends out of the base body 11 to achieve some specific purposes, for example degassing and/or air separating and/or disinfecting, which may require a specific fluid flow direction transverse to the plane of the base body 11. That is to say, such a vertical fluid cavity 111 is essential for the specific purposes and thus a fluid flow direction in the vertical fluid cavity 111 cannot be changed arbitrarily.

It may be understood by the skilled person in the art that the standing structure 12 should not be regarded as any protruding structure as the base body 11 itself may comprise some protruding structures. For example, the standing structure 12 should extend upwards from the base body 11 by a certain height, for example not less than 10 mm.

In some embodiments, as shown in FIG. 1 and FIG. 2, the standing structure 12 stands from a main face of the base body 11, not a lateral edge of the base body 11, e.g., in the case that the base body 11 is configured as a flat body, for example in a plate shape. Thus, in this case, any tube joint disposed at the lateral edge of the base body 11 should not be regarded as the standing structure 12.

According to an exemplary embodiment of the present disclosure, as shown in FIG. 2, the fluid accommodating cavity 111 may open initially at a side, e.g., facing away from the standing structure 12, of the base body 11 after molding of the standing structure 12 and the base body 11. The open fluid accommodating cavity 111 can be closed as desired, which will be further described below.

As shown in FIG. 2, the fluid accommodating cavity 111 may comprise a flow path, which usually has a relatively low width. That is to say, the flow path may be formed directly in the base body 11.

According to an exemplary embodiment of the present disclosure, the hydraulic block 1 may be configured to prepare and/or deliver dialysate. It may be understood by the skilled person in the art that on-site preparation of the dialysate is very advantageous.

In some embodiments, the hydraulic block I may be configured to be reusable, e.g., after being disinfected, which will reduce significantly its usage cost. In this case, the disinfectant and/or degreasing agent can be introduced into the hydraulic block 1 for cleaning purpose.

According to an exemplary embodiment of the present disclosure, the standing structure 12 may be configured as at least one of a water inlet chamber, a heating chamber for heating water, a degassing chamber for water, and an air separation chamber for used dialysate. For preparing the dialysate, the water inlet chamber may be provided to receive fresh water, such as reverse osmosis water. The water possibly contains air bubbles so that the degassing chamber may be necessary to release the air bubbles from the water. The water can be heated by the heating chamber, if necessary. The used dialysate also possibly contains air that needs to be removed by the air separation chamber before returning to a balancing chamber to achieve a balancing function, which is known in the art and thus is not described in details here.

It may be understood by the skilled person in the art that if the standing structure and the base body are molded individually, the present disclosure also relates to such a molded standing structure comprising at least one vertical fluid cavity and configured to be fitted onto the base body in a standing manner so that the at least one vertical fluid cavity is fluidly connected with the fluid accommodating cavity formed in the base body. In this case, the standing structure may be at least one of the water inlet chamber, the heating chamber for heating water, the degassing chamber for water, and the air separation chamber for used dialysate, as mentioned above.

Further, the present disclosure relates to such a molded base body formed with a fluid accommodating cavity and configured to allow for fitting a standing structure thereon in a standing manner so that the fluid accommodating cavity is fluidly connected with the at least one vertical fluid cavity formed in the standing structure.

According to an exemplary embodiment of the present disclosure, the standing structure 12 and the base body 11 may be molded from at least one of PES, PPO and PPSU, which have good chemical resistance and heat resistance so that the hydraulic block 1 can be disinfected and reused even during the whole lifetime of the HD machine. Of course, it may be understood by the skilled person in the art that the standing structure 12 and the base body 11 also may be made from any other suitable materials.

According to an exemplary embodiment of the present disclosure, the standing structure 12 and the base body 11 may be molded by a one-spot injection molding process.

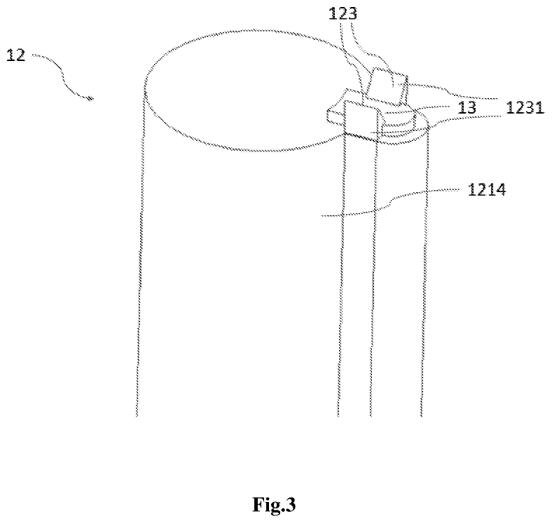

FIG. 3 shows a perspective view of the standing structure 12 according to an exemplary embodiment of the present disclosure, which is fitted with a sealing structure 13, which will be further described below.

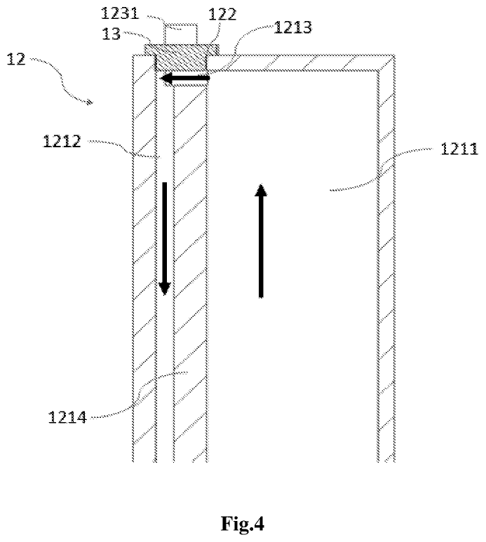

FIG. 4 shows a sectional view of the standing structure 12 as shown in FIG. 3, with the sealing structure 13.

According to an exemplary embodiment of the present disclosure, as shown in FIG. 4, the at least one vertical fluid cavity 121 may comprise at least one vertical fluid chamber 1211 and at least one vertical flow channel 1212 which are in fluid communication with each other via a fluid communication path 1213.

In some embodiments, the fluid communication path 1213 may be molded at a top of the standing structure 12 so as to allow fluid to flow into or out of a top of the vertical fluid chamber 1211 via the vertical flow channel 1212. The vertical fluid chamber 1211 may have a larger flow cross-section than the vertical flow channel 1212.

For example, in use, the fluid may flow upward in the vertical fluid chamber 1211 and then flow into a top of the vertical flow channel 1212 via the fluid communication path 1213, as shown by arrows in FIG. 4. In this case, the standing structure 12 can be used as the degassing chamber for water or the air separation chamber for the used dialysate. When being used as the air separation chamber, the at least one vertical flow channel 1212 may comprise two vertical flow channels 1212, e.g., adjacent to each other, as shown in the top right corner of FIG. 1.

As shown in FIG. 1, according to an exemplary embodiment of the present disclosure, the base body 11 may be molded with at least one installation interface 112 for at least one functional component (not shown in FIG. 1) for dialysis. In some embodiments, the at least one functional component may comprise at least one of tubes, pumps, valves and sensors, which are required for achieving a desired function of the hydraulic block.

According to an exemplary embodiment of the present disclosure, the installation interface 112 may be configured as a protruding seat, e.g., lower than the standing structure 12.

In some examples, the installation interface 112 may be configured as a quick connector, which will facilitate mounting of the functional component onto the base body 11.

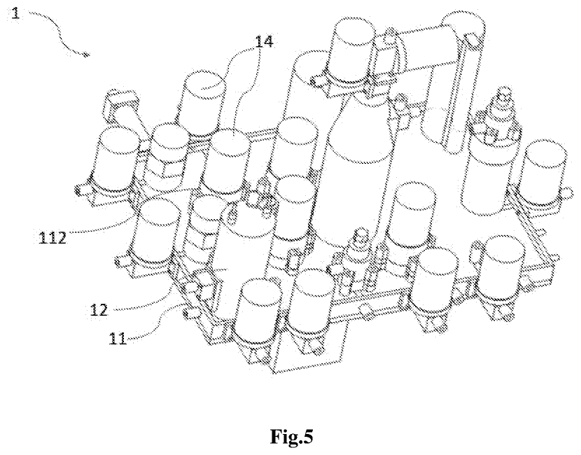

FIG. 5 shows a perspective view of the hydraulic block 1 when the functional components 14 have been mounted on the installation interfaces 112.

As described above, the fresh dialysate may be prepared by means of the hydraulic block 1, and thus the base body 11 may be molded with a mixing structure 113 for preparing dialysate, as can be seen from FIG. 2.

As a possible embodiment, the mixing structure 113 may be molded individually and then fitted onto the base body 11. In this case, the present disclosure relates to a mixing structure molded as a piece.

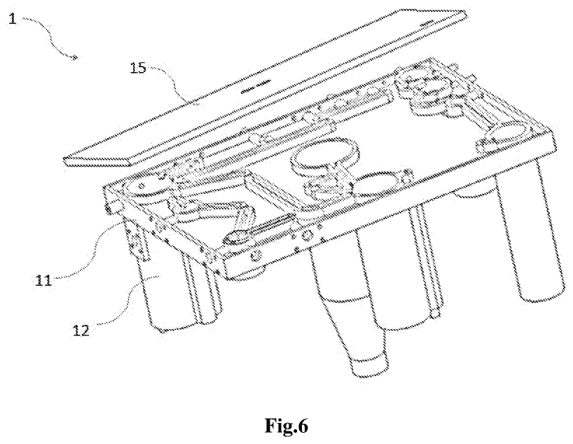

According to an exemplary embodiment of the present disclosure, the hydraulic block 1 may comprise a cover 15 fixedly connected to the base body 11 to close the fluid accommodating cavity 111 and/or the mixing structure 113. FIG. 6 shows a perspective view of the hydraulic block 1 when the cover 15 is to be fixedly connected to the base body 11. The cover 15 can be a plastic cover.

In some embodiments, the mixing structure 113 is open at the same side as the fluid accommodating cavity 111 and is closed by the same cover 15,

If the functional component is a pump, it may be mounted at a side of the cover 15. According to an exemplary embodiment of the present disclosure, the cover 15 may be bonded to the base body 11 by a plastic bonding process, for example at least one of an ultrasonic welding process, a diffusion bonding process, an infrared welding process, a resistive welding process and a laser welding process. However, it may be understood by the skilled person in the art that the cover 15 also may be connected to the base body 11 by any other suitable processes.

The laser welding process can achieve a high bonding strength and have high efficiency and thus is an advantageous solution. However, because of mechanical assembling or manufacturing tolerance and strain of the welded parts, it is hard to ensure that big planar surfaces to be welded can contact seamlessly with each other, which will lead to cold joint and lower bonding strength and in turn lead to leakage. Moreover, during the laser welding process, material (e.g., plastic) to be welded will be melted and flow violently in the welding pool. The melted material may be splashed out and finally get solidified in the fluid accommodating cavity 111, which will adversely affect or even block the fluid accommodating cavity 111, e.g., when the fluid accommodating cavity 111 is the narrow flow path.

Thus, it is advantageous to take technical measures to address the above-mentioned laser welding issues.

According to an exemplary embodiment of the present disclosure, a welding structure may be provided to facilitate welding, e.g., laser welding, between the cover 15 and the base body 11.

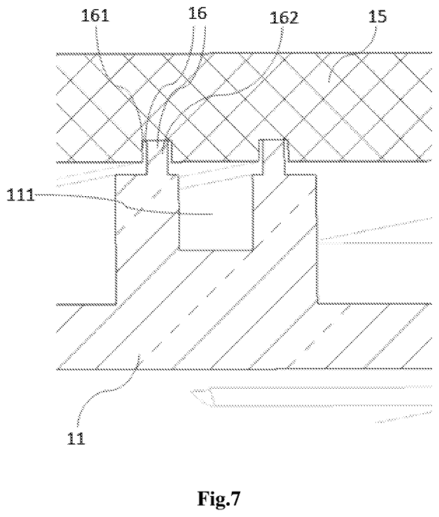

FIG. 7 shows a sectional view for illustrating the welding structure 16 according to an exemplary embodiment of the present disclosure. For facilitating welding, the cover 15 may be formed at least partially from a material transparent to laser. However, for absorbing the laser, the base body 11 may be formed at least partially from a non-transparent material.

As shown in FIG. 7, the welding structure 16 may comprise a first welding portion 161, for example one of a groove and a rib, formed at a site to be welded of the cover 15, and a second welding portion 162, for example the other of the groove and the rib, formed at a site to be welded of the base body 11 and configured to cooperate with the first welding portion 161 to form a welding seam. Cooperation of the rib and the groove can allow for controlling the welding pool during the welding process and avoiding excessive melted plastic flowing into the fluid accommodating cavity 111, e.g., the flow path and forming some flaws such as burs. In this case, the welding seam can be formed in a predefined manner.

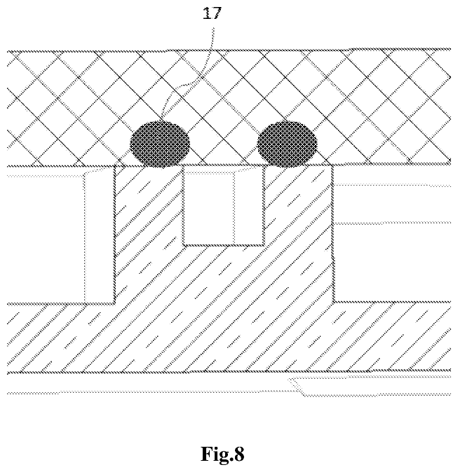

FIG. 8 shows a sectional view for illustrating the welding structure 16 as shown in FIG. 7 after welding, wherein the welding seam 17 is formed so as to securely fix the cover 15 to the base body 11.

According to an exemplary embodiment of the present disclosure, as shown in FIG. 7, the first welding portion 161 may be configured as the groove and the second welding portion 162 may be configured as the rib.

According to an exemplary embodiment of the present disclosure, as shown in FIG. 7, a height of the rib may be greater than a depth of the groove, for example by a 0.5-1 mm, before welding, so that the manufacturing or assembling tolerance between the cover 15 and the base body 11 can be compensated. According to an exemplary embodiment of the present disclosure, as shown in FIG. 7, the rib and the groove may be located adjacent to an edge to be sealed of the fluid accommodating cavity 111. It may be understood by the skilled person in the art that the rib and the groove closely adjacent to the fluid accommodating cavity 111 will result in no gap between the two welded parts, as shown in FIG. 8.

The rib and/or the groove may be configured to have a rectangular cross-sectional shape. In some embodiments, the groove may have a slightly larger width than the rib.

Referring back to FIG. 3 and FIG. 4, according to an exemplary embodiment of the present disclosure, the vertical flow channel 1212 may be located outside of the vertical fluid chamber 1211 and adjacent to a vertical wall 1214 of the vertical fluid chamber 1211. The vertical fluid chamber 1211 and the vertical flow channel 1212 may be located side by side.

In some embodiments, as shown in FIG. 3 and FIG. 4, the vertical flow channel 1212 may be partially defined by a corresponding portion of the vertical wall 1214 of the vertical fluid chamber 1211. That is to say, a portion of the vertical flow channel 1212 may be formed directly from the corresponding portion of the vertical wall 1214 of the vertical fluid chamber 1211.

According to an exemplary embodiment of the present disclosure, the vertical flow channel 1212 may be molded onto the vertical fluid chamber 1211, as shown in FIG. 3 and FIG. 4.

As shown in FIG. 4, the vertical fluid chamber 1211 may have a larger cross-sectional area than the vertical flow channel 1212. Also, the vertical fluid chamber 1211 and/or the vertical flow channel 1212 may have a circular cross-section.

According to an exemplary embodiment of the present disclosure, the vertical fluid chamber 1211 may be molded by a first columnar core (not shown), for example a cylindrical core, on a mold (not shown). In some embodiments, the first columnar core may have a draft angle of 1-3 degrees.

Similarly, the vertical flow channel 1212 may be molded by at least one second columnar core, for example a cylindrical core, on the mold. In some embodiments, the second columnar core may have a draft angle of about 0.5 degrees.

According to an exemplary embodiment of the present disclosure, the vertical fluid chamber 1211 may be molded by positioning the first columnar core on one side of the mold.

Similarly, the vertical flow channel 1212 may be molded by positioning one second columnar core on each of two sides of the mold.

According to an exemplary embodiment of the present disclosure, the second columnar cores may be connected at a middle position of the vertical flow channel 1212 to be molded.

As shown in FIG. 3, according to an exemplary embodiment of the present disclosure, the standing structure 12 may be molded with an outward opening 122 directly leading to the fluid communication path 1213.

As shown in FIG. 4, according to an exemplary embodiment of the present disclosure, the outward opening 122 may be oriented upwards.

According to an exemplary embodiment of the present disclosure, the outward opening 122 may be configured to be closeable, as shown FIG. 3 and FIG. 4. If necessary, the outward opening 122 also may be fluidly connected with an external part (not shown).

In some embodiments, the outward opening 122 may be closed by the sealing structure 13, e.g., a cap which can be mounted or bonded at the outward opening 122.

According to an exemplary embodiment of the present disclosure, the cap may be configured to be mounted in a form-fitting manner, for example in a snap-fitting manner, and/or by using a fastener, for example a screw, or to be bonded by welding.

As shown in FIG. 3 and FIG. 4, the cap may be fixed onto the standing structure 12 by a snap-fit structure 123 molded integrally with the standing structure 12. The snap-fit structure 123 may comprise two clamp legs 1231 facing toward to each other, between the cap can be clamped to close the outward opening 122.

The clamp leg 1231 may have a barb-like shape so that the cap can be firmly fixed at the outward opening 122.

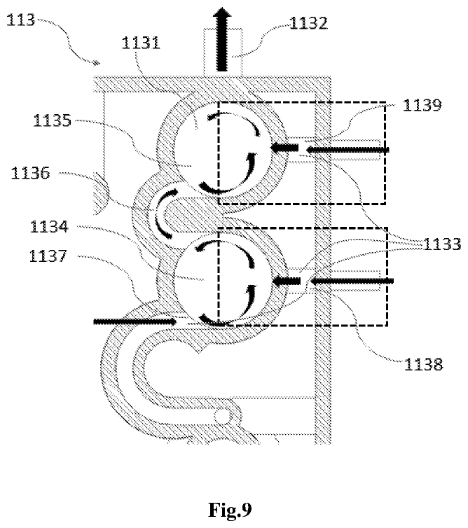

FIG. 9 shows a sectional view for illustrating the mixing structure 113 according to an exemplary embodiment of the present disclosure.

As shown in FIG. 9 (also possibly in connection with FIG. 2), the mixing structure 113 may comprise at least one mixing chamber 1131, a fluid outlet 1132 opening into the mixing chamber 1131, and at least two fluid inlets 1133 each opening into the mixing chamber 1131.

In FIG. 9, some arrows also are used to show schematically flow directions of fluids during a mixing process. The skilled person in the art may better understand mixing operation and thus arrangement of the mixing structure 113 by means of these arrows.

According to an exemplary embodiment of the present disclosure, as shown in FIG. 9, the at least one mixing chamber 1131 may comprise a first chamber 1134 and a second chamber 1135 fluidly connected to the first chamber 1134 by a flow communication passage 1136 and located downstream of the first chamber 1134, and the at least two fluid inlets 1113 may comprise a first fluid inlet 1137 for a first fluid, for example water, e.g., reverse osmosis water, a second fluid inlet 1138 for a second fluid, and a third fluid inlet 1139 for a third fluid, wherein the first fluid inlet 1137 may open tangentially into the first chamber 1134, the second fluid inlet 1138 may open into the first chamber 1134, the third fluid inlet 1139 may open into the second chamber 1135, and the fluid outlet 1132 may open into the second chamber 1135.

According to an exemplary embodiment of the present disclosure, as shown in FIG. 9, the second fluid inlet 1138 may be located downstream of the first fluid inlet 1137.

According to an exemplary embodiment of the present disclosure, as shown in FIG. 9, the second fluid inlet 1138 is oriented toward a center of the first chamber 1134.

Similarly, the third fluid inlet 1439 and/or the fluid outlet 1132 may be oriented toward a center of the second chamber 1135.

As shown in FIG. 9, the first chamber 1134 and/or the second chamber 1135 may be configured as a circular chamber.

According to an exemplary embodiment of the present disclosure, as shown in FIG. 9, the mixing structure 113 may be configured so that a first swirling fluid flow can be generated within the first chamber 1134, as shown schematically by two corresponding arrows.

Similarly, the mixing structure 113 may be configured so that a second swirling fluid flow can be generated within the second chamber 1135, also as shown schematically by two corresponding arrows.

According to an exemplary embodiment of the present disclosure, as shown in FIG. 9, the flow communication passage 1136 may be configured in an arc shape.

As shown in FIG. 9, the first swirling fluid flow and the second swirling fluid flow may have the same swirling direction.

According to an exemplary embodiment of the present disclosure, as shown in FIG. 9, the flow communication passage 1136 may be configured to be bent outwards. Specifically, the flow communication passage 1136 may be outwardly convexly curved.

According to an exemplary embodiment of the present disclosure, as shown in FIG. 9, the mixing structure 113 may be configured so that a first fluid flow direction into the first chamber 1134 via the first fluid inlet 1137 is opposite to a second fluid flow direction into the first chamber 1134 via the second fluid inlet 1138, which will lead to dramatic mixing within the first chamber 1134, as shown schematically by corresponding arrows.

According to an exemplary embodiment of the present disclosure, as shown in FIG. 9, the flow communication passage 1136 may open tangentially into the first chamber 1134. Similarly, the flow communication passage 1136 also may open tangentially into the second chamber 1135

According to an exemplary embodiment of the present disclosure, as shown in FIG. 9, the mixing structure 113 may be configured so that a third fluid flow direction into the flow communication passage 1136 from the first chamber 1134 is opposite to the first fluid flow direction into the first chamber 1134 via the first fluid inlet 1137 and/or parallel to the second fluid flow direction into the first chamber 1134 via the second fluid inlet 1138.

According to an exemplary embodiment of the present disclosure, as shown in FIG. 9, the mixing structure 113 may be configured so that a fourth fluid flow direction into the second chamber 1135 via the flow communication passage 1136 is opposite to the third fluid flow direction into the flow communication passage 1136 from the first chamber 1134 and/or parallel to a fifth fluid flow direction into the second chamber 1135 via the third fluid inlet 1139.

According to an exemplary embodiment of the present disclosure, as shown in FIG. 9, the mixing structure 113 may be configured so that a sixth fluid flow direction out of the second chamber 1135 via the fluid outlet 1132 is substantially perpendicular to the fifth fluid flow direction into the second chamber 1135 via the third fluid inlet 1139.

According to exemplary embodiment of the present disclosure, at least one of the second fluid and the third fluid may be concentrate required for preparing dialysate, for example bicarbonate. Mixing and/or dissolving of the concentrates with the first fluid can produce the dialysate which can flow out of the second chamber 1135 via the fluid outlet 1132 and then for example flow toward a dialyzer (not shown).

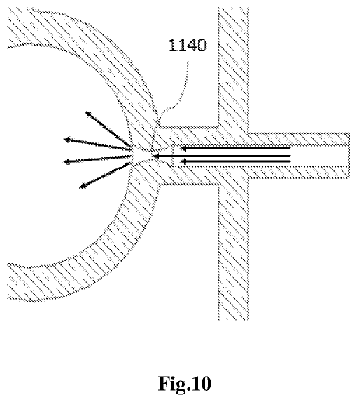

FIG. 10 shows a partial sectional view of one of portions selected by dotted boxes as shown in FIG. 9.

According to exemplary embodiment of the present disclosure, as shown in FIG. 10, the second fluid inlet 1138 and/or the third fluid inlet 1139 may be molded with a narrowed orifice 1140, which can create a fluid ejecting to facilitate mixing, as shown schematically by some arrows.

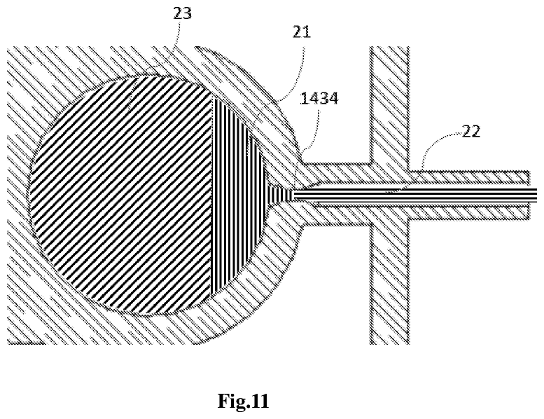

FIG. 11 schematically shows how to mold the first chamber 1134 and/or the second chamber 1135 in the case that the second fluid inlet 1138 and/or the third fluid inlet 1139 is molded with the corresponding narrowed orifice 1140. According to exemplary embodiment of the present disclosure, the narrowed orifice 1140 may be molded by a first slider 21 located at a first side (i.e., the left side in FIG. 11) of the narrowed orifice 1140 facing toward the first chamber 1134 or the second chamber 1135 and a second slider 22 located at a second side (i.e., the right side in FIG. 11) of the narrowed orifice 1140 opposite to the first side.

As shown in FIG. 11, at least one of the first slider 21 and the second slider 22 may have a tapered end for molding narrowed orifice 1140.

Further, as shown in FIG. 11, the first slider 21 may partially extend into the first chamber 1134 or the second chamber 1135. In this case, the first chamber 1134 and/or the second chamber 1135 may be molded by the first slider 21 and a chamber molding core 23 cooperating with the first slider 21. That is to say, the chamber molding core 23 and a portion of the first slider 21 together are used to mold the first chamber 1134 or the second chamber 1135.

During a demolding process, the chamber molding core 23 may be drawn in a drawing direction (for example, perpendicular to the plane of FIG. 11) and then the first slider 21 may be pulled away in a pulling direction (for example, the leftward direction in FIG. 11) different from, e.g., perpendicular to, the drawing direction. The second slider 22 may be pulled away in an opposite pulling direction.

According to a second aspect of the present disclosure, further provided is a hydraulic system for dialysis, wherein the hydraulic system may comprise the hydraulic block 1 described above and at least one functional component 14 mounted on the hydraulic block 1.

According to a third aspect of the present disclosure, further provided is a method for manufacturing the hydraulic block 1 describe above, wherein the method comprises molding the hydraulic block by using a mold.

While certain embodiments have been described, these embodiments have been presented by way of example only, and are not intended to limit the scope of the present disclosure. The attached claims and their equivalents are intended to cover all the modifications, substitutions and changes as would fall within the scope and spirit of the present disclosure.

1-21. (canceled)

22. A hydraulic block for dialysis, comprising:
a base body formed with a fluid accommodating cavity; and
at least one standing structure standing on the base body, wherein the standing structure comprises at least one vertical fluid cavity fluidly connected with the fluid accommodating cavity.

23. The hydraulic block according to claim 22, wherein the standing structure and the base body are molded integrally; or
the standing structure and the base body are molded individually so that the standing structure can be fitted onto the base body.

24. The hydraulic block according to claim 22, wherein at least one standing structure of the at least one standing structure is molded individually, and the base body and the rest of the at least one standing structure are molded integrally, so that the at least one standing structure can be fitted onto the base body.

25. The hydraulic block according to claim 22, wherein the fluid accommodating cavity initially opens at a side of the base body after molding of the base body.

26. The hydraulic block according to claim 22, wherein the fluid accommodating cavity comprises a flow path.

27. The hydraulic block according to claim 22, wherein the at least one standing structure is configured as at least one of a water inlet chamber, a heating chamber for heating water, a degassing chamber for water, and an air separation chamber for a used dialysate.

28. The hydraulic block according to claim 22, wherein the base body is configured as a flat body.

29. The hydraulic block according to claim 22, wherein the at least one standing structure and the base body are molded as a single plastic piece.

30. The hydraulic block according to claim 22, wherein the at least one vertical fluid cavity comprises at least one vertical fluid chamber and at least one vertical flow channel which are in fluid communication with each other via a fluid communication path.

31. The hydraulic block according to claim 22, wherein the base body is molded with at least one installation interface for at least one functional component for dialysis.

32. The hydraulic block according to claim 31, wherein the base body is molded with or fitted with a mixing structure for preparing dialysate.

33. The hydraulic block according to claim 32, wherein the hydraulic block comprises a cover fixedly connected to the base body to close the fluid accommodating cavity.

34. The hydraulic block according to claim 33, wherein the vertical flow channel is partially defined by a corresponding portion of the vertical wall of the vertical fluid chamber.

35. The hydraulic block according to claim 33, wherein the vertical fluid chamber is molded by a first columnar core comprising a first cylindrical core.

36. The hydraulic block according to claim 35, wherein the vertical flow channel is molded by at least one second columnar core comprising at least one second cylindrical core.

37. The hydraulic block according to claim 33, wherein the standing structure is molded with an outward opening directly leading to the fluid communication path, and wherein the outward opening is configured to be closeable or to be fluidly connected with an external part.

38. The hydraulic block according to claim 33, wherein the mixing structure comprises at least one mixing chamber, a fluid outlet-opening into the mixing chamber, and at least two fluid inlets-each opening into the mixing chamber.

39. The hydraulic block according to claim 38, wherein the vertical flow channel is located outside of the vertical fluid chamber and adjacent to a vertical wall of the vertical fluid chamber.

40. The hydraulic block according to claim 39, wherein the vertical flow channel is molded onto the vertical fluid chamber.

41. The hydraulic block according to claim 50, wherein the vertical fluid chamber is molded by positioning a first columnar core on one side of the mold.
